(19) United States
(12) Patent Application Publication
(10) Pub. No.: US 2010/0009396 A1
(43) Pub. Date: Jan. 14, 2010

(54) METHOD FOR MEASURING THE CONCENTRATION OF ACTIVATED FACTOR VII (FVIIA) IN A SAMPLE

(76) Inventors: Lysiane Hilbert, Villeneuve- d'Ascq (FR); Dominique Grenier, Hellemmes (FR); Claudine Mazurier, Villeneuve-d'ascq (FR)

Correspondence Address: HARNESS, DICKEY & PIERCE, P.L.C., P.O. BOX 828, BLOOMFIELD HILLS, MI, 48303, US

(21) Appl. No.: 12/520,019

(22) PCT Filed: Dec. 28, 2007
(86) PCT No.: PCT/FR07/02188

§ 371 Date: Jun. 18, 2009

(30) Foreign Application Priority Data
Dec. 29, 2006 (FR) 06/11535

(51) Int. Cl. C12Q 1/56; C12N 5/00

(52) U.S. Cl. 435/13; 435/325

(57) ABSTRACT

The present application concerns a method for the in vitro or ex-vivo measurement of the FVIIa concentration of a sample, comprising the steps consisting of:
a) mixing said sample with a human plasma deficient in FVII and in at least one other factor chosen from among FVIII, FIX and FXI;
b) adding initiator components of the thrombin generation reaction, comprising a source of calcium ions, a phospholipid agent and tissue factor;
c) performing a thrombin generation test (TGT) on the reaction medium so obtained, to obtain a thrombogram giving parameters;
d) comparing at least one of the parameters of the thrombogram with a homologous parameter of standard thrombograms, each standard thrombogram being obtained with a fixed, calibrated concentration of FVIIa in said reaction medium, lying a range of 1 pM to 5 nM, and
e) deducing from step d) the measurement of the FVIIa concentration of the sample, lying within said range.

### CROSS-REFERENCE TO RELATED APPLICATIONS

This application is a National Phase Entry of International Application No. PCT/FR2007/002188, filed Dec. 28, 2007, which claims priority to French Patent Application No. 06 11 535, filed Dec. 29, 2006, both of which are incorporated herein by reference.

### BACKGROUND AND SUMMARY

The present invention relates to a method to measure the concentration of activated factor VII (FVIIa) in a sample, using a plasma deficient in factor VII (FVII) and in at least one other factor chosen from among factor VIII (FVIII), factor IX (FIX) and factor XI (FXI).

Blood coagulation is a mechanism which enables the body to control bleeding in the event of vascular injury, and hence to avoid haemorrhages. Blood coagulation occurs in steps, in a cascade, involving different proenzymes and procofactors present in the blood which, via proteolytic enzymes, are converted to their activated form. In this succession of steps (or cascade) of coagulation, two pathways can be distinguished termed the extrinsic coagulation pathway and the intrinsic coagulation pathway. Both lead to the formation of the complex called prothrombinase, consisting of activated factor X (FXa), activated factor V (FVa), phospholipids and calcium. It is prothrombinase which activates prothrombin to thrombin, enabling the conversion of soluble fibrinogen to insoluble fibrin which forms the clot.

The extrinsic pathway involves the action of FVII present in the plasma. However, it must previously be activated to FVIIa in order to initiate the coagulation cascade. FVIIa alone (not complexed) has low proteolytic activity. This activity is potentialized when FVIIa is complexed with the tissue factor (TF), a protein associated with phospholipids, which is released in the event of vascular injury. The FVIIa-TF complex converts factor X to factor Xa in the presence of calcium ions. The FVIIa-TF complex also converts FIX to FIXa, thereby catalyzing the intrinsic pathway of coagulation. In return, factors IXa and Xa activate FVII to FVIIa. Factor Xa, complexed with factor Va and with the phospholipids (prothrombinase) converts prothrombin to thrombin. Thrombin acts on fibrinogene converting it to fibrin, and also has other activities amongst which activation of factor V to factor Va and of FVIII to FVIIIa. Thrombin, in the presence of calcium, also activates factor XIII to factor XIIIa which allows consolidation of the fibrin clot.

While in the extrinsic pathway of coagulation FIX is activated to FIXa by the FVIIa/TF complex, in the intrinsic coagulation pathway FIXa is generated from FIX by FXIa, which itself is activated by factor XII activated by contact of the blood with an electronegative surface such as the sub-endothelium. FVIIa, a glycoprotein dependent on vitamin K, therefore plays an important role in the mechanisms of coagulation, leading to blood clot formation. FVIIa has the advantage that it can act locally in the presence of the tissue factor, released after tissue injury generating haemorrhages, even in the absence of Factor VIII or IX. This is why FVIIa has been used for many years to correct some coagulation disorders translating as bleeding.

The first approach was to obtain FVIIa from the plasma. However, the production of FVIIa from plasma is limited by the availability of the supply source, and this use of plasma carries risks of transmitting pathogenic agents such as the prion for example and viruses. These problems were solved by NovoNordisk Pharmaceuticals with the development of a recombinant FVIIa (rFVIIa) which is a glycoprotein structurally similar to plasma FVIIa. The chief therapeutic indication for rFVIIa (in the USA, EU and Japan) is the treatment of spontaneous or surgically-induced bleeding in haemophilia A individuals who have developed anti-factor VIII antibodies, and in haemophilia B individuals who have developed anti-factor IX antibodies. In Europe, it is also indicated for use in patients with congenital FVII deficiency, and in patients suffering from Glanzmann's thrombasthenia.

In addition, numerous publications report on the efficacy of rFVIIa to control haemorrhages at surgery in patients who have neither congenital deficiency of the coagulation factor nor thrombasthenia. This increasingly wider use of FVIIa has led to the development of methods to measure the activity of FVIIa, and methods to determine FVIIa concentration e.g. to measure the concentration of a sample potentially containing FVIIa, or to measure the plasma concentration of FVIIa in a patient in order to verify that the administered dose of FVIIa is well adapted to the treatment of the haemostasis disorder to be corrected, and that the treated patient is not in a state of hypo-coagulation or hyper-coagulation.

The most known methods to detect FVIIa activity are the measurement of coagulation time, PTT (Partial Thromboplastin Time), aPTT (activated partial thromboplastin time), TEG (thromboelastography) and TGT (thrombin generation test). With these methods it is possible to measure the activity of FVIIa, but not to measure directly the precise concentration of FVIIa. Direct measurements of FVIIa concentration, such as fluorogenic or chromogenic assay of FXa generated by FVIIa, have proved to be unsuitable since they do not allow a differentiation to be made between the effects of FVIIa and FVII.

A FVIIa immunoassay kit is available on the market (IMUBIND Factor VII ELISA kit), but the experimental conditions for implementing this technique are difficult to control. Other methods to measure FVIIa concentration are described in the literature, such as the measurement of its proteolytic activity with the use of recombinant truncated TF (Staclot VIIa-rTF, Stago) (U.S. Pat. No. 5,472,850, U.S. Pat. No. 5,741,658, WO 1992/018870, U.S. Pat. No. 5,750,358, U.S. Pat. No. 5,741,658, U.S. Pat. No. 5,472,850, EP 0 641 443) or measurement of the concentration of a FVIIa-antithrombin complex (WO 03/004694). However, these methods are not very precise and are difficult to implement.

At the current time, one method frequently used by some biologists and clinicians to assess the efficacy of a treatment with FVIIa, is based on the use of thromboelastography. This method consists of measuring the physical properties of whole blood by mechanical analysis of clot formation in relation to time. According to the parameters extracted from a graph (called a thrombogram or thromboelastogram®) generated by the thrombolastograph®, the clinician can assess a patient's coagulation capability. Although accurate, this method is tedious, scarcely adapted for repetitive routine analysis and difficult to apply to multisampling since it has to be conducted within one hour after the blood sample is taken. In addition, this method does not allow FVIIa concentration to be measured. There is therefore a real need for an efficient method, easy to implement, to measure FVIIa concentration, for example to measure the concentration of a sample potentially containing FVIIa, or to measure a patient's plasma concentration of FVIIa to check that the therapeutic dose of FVIIa received is well adapted to the haemostasis disorder to be treated, and that the treated patient is not in a state of hypo-coagulation or hyper-coagulation, as indicated above.

The present invention concerns a method for the in vitro or ex-vivo measurement of FVIIa concentration in a sample, comprising the steps consisting of:

a) mixing said sample with a human plasma deficient in FVII and in at least one other factor chosen from among FVIII, FIX and FXI;

b) adding components initiating the thrombin generation reaction, comprising a source of calcium ions, a phospholipid agent and tissue factor;

c) performing a thrombin generation test (TGT) on the reaction medium thus obtained, to plot a thrombogram giving parameters;

d) comparing at least one of the parameters of the thrombogram with a homologous parameter of standard thrombinograms, each standard thrombogram being obtained with a fixed calibrated concentration of FVIIa in said reaction medium, lying in a range of between 1 pM to 5 nM, and

e) deducing from step d) the measurement of the FVIIa concentration of the sample, lying within said range.

The Applicant has found, in surprising manner, that the use of a plasma deficient in FVII and in at least one other factor chosen from among FVIII, FIX and FXII (doubly depleted plasma) makes it possible to obtain a reagent which can be used in a method to measure the FVIIa concentration of a sample, that is reliable, reproducible and easy to use. With the method of the invention it is now possible to establish a correlation between the FVIIa concentration of a sample and certain parameters of a thrombin generation test.

By means of this correlation determined using standard thrombograms i.e. plotted using variable, calibrated concentrations of FVIIa in the reaction medium, it is possible to determine the unknown concentration of FVIIa in a sample, by comparison of data given by standard thrombograms with data given by the thrombogram of the sample to be measured. The thrombin generation test (TGT) of step c), known to those skilled in the art, is based on continuous measurement of quantities of generated thrombin and the time required to generate the thrombin when the initiator components of the thrombin generation reaction, in this case the source of calcium ions, phospholipid agent and tissue factor (TF), are contacted with the sample containing FVIIa mixed with a plasma deficient in FVII and in at least one other factor chosen from among FVIII, FIX and FXI.

As soon as all these components, which therefore form the reaction medium, and of predetermined volume, are placed in contact, the thrombin generation reaction is initiated, thereby making it possible to determine the start time of the test (t₀). Evidently, the content of each of the components is chosen so that this reaction is able to occur. This test is advantageously implemented using an agent to develop the generated thrombin. Said agent is a fluorogenic agent, which will be degraded by the thrombin to yield a fluorescence-emitting compound, or a chromogenic agent.

Fluorescence is detected by a device to measure the formation of thrombin in the reaction medium, such as a conventional fluorimeter also comprising software able to collect data which can be used to plot a thrombogram. This thrombogram shows a curve, traced in relation to test duration, having a maximum corresponding to the maximum quantity of thrombin generated. The greater the increase in the quantity of FVIIa in the sample, and hence in the reaction medium, the shorter the thrombin generation time, decreasing down to a limit value.

The thrombogram can provide data representing at least one of the parameters of the thrombogram (step d)) defined as follows. The first parameter is peak height which corresponds to the maximum thrombin generated, as indicated above. The second is lag time corresponding to the time lapsed between the start of the TGT test (t₀) and the onset of thrombin. Time to peak, the third parameter, corresponds to the time lapsed between the start of the TGT test (t₀) and the maximum thrombin generated. Velocity, the fourth parameter, expressed in nM/min of thrombin formed, corresponds to the peak height divided by the difference between time to peak and lag time. These parameters can be provided directly by the device measuring thrombin formation. Therefore, at step d), at least one of the thrombogram parameters obtained with the method of the invention is chosen from among peak height, velocity, lag time and time to peak.

According to the invention, the parameters of standard thrombograms are determined by conducting a series of TGT tests with calibrated samples, consisting of the same reaction medium, but each thereof comprises a final, fixed calibrated concentration of FIIVa in the range of 1 pM to 5 nM, preferably in the range of 1 pM to 1 nM for the parameters chosen from among peak height and velocity, and preferably in the range of 1 pM to 5 nM for the four parameters. The same operating conditions are therefore used to maintain one same volume of reaction medium, although slight variations in volume are acceptable through the addition of the FVIIa sample. A thrombogram is therefore obtained for each reaction medium whose FVIIa content is calibrated.

In one embodiment of the invention, the FVIIa used to establish standard thrombogram parameters is an international FVIIa standard (IS-FVIIa), made available by the National Institute for Biological Standard and Control in England (NIBSC). From each standard thrombogram, calibrated variation curves are determined for each of the above parameters in relation to the final concentration of FVIIa in the reaction medium. The above parameters obtained for a sample whose FVIIa concentration is unknown, are then compared with homologous parameters obtained previously (step d)). In this manner the FVIIa concentration of the measured sample can be inferred directly, irrespective of the parameter used, since this concentration is determined by comparison with calibrated standard concentration curves i.e. determined using standard thrombograms. Under the invention, the FVIIa sample, the plasma deficient in FVII and in at least one other factor chosen from among FVIII, FIX or FXI, the components initiating the thrombin generation reaction, can be in liquid or lyophilized form, the reaction medium then being prepared by dissolution in a suitable aqueous solvent, such as water for injection (WFI).

According to one particular embodiment of the invention, the FVIIa sample is in liquid form. To plot standard thrombograms, all that is required is to sample the appropriate volume to be mixed with the doubly depleted plasma under consideration, in order to obtain the desired final concentrations in the reaction medium. This can also be done by adding a constant volume of the FVIIa sample to said plasma, but of different concentrations. It is also possible to plot several thrombograms for a sample having a FVIIa content to be determined, by successive dilutions thereof and addition of one same volume to the doubly depleted plasma under consideration, the volume of reaction medium being kept constant. By comparison of the results of the parameters obtained with those of standards, and with knowledge of the dilution ratios, it is possible to find the FVIIa content with accuracy.

Any phospholipid agent is suitable for the invention. It may be in the form of a concentrate or lyophilisate. Preferably, it is in the form of a mixture mainly or solely containing phosphatidylcholine and phosphatidylserine.

Similarly, any tissue factor (TF), native, plasma, recombinant or transgenic TF is suitable. Additionally, some modified TFs, in particular any truncated TF which has lost its function enabling it to convert factor VII to factor VIIa whilst maintaining its capability to act as cofactor for the enzymatic activity of factor VIIa, is suitable. In particular, said TF has its transmembrane domain deleted, this deletion allowing the desired selective deficit to be obtained in the TF function (said tissue factor is easily commercially available). In one embodiment of the invention, said modified TF is used for in vitro or ex-vivo measurement of the FVIIa concentration of a sample also containing FVII.

According to one embodiment of the invention, the sample containing FVIIa is a therapeutic sample or not, containing plasma FVIIa (pFVIIa), recombinant FVIIa (rFVIIa) or transgenic FVIIa (TgFVIIa), in liquid or lyophilized form. In one particular embodiment, the sample containing FVIIa is a sample of mammalian milk, in particular a sample of transgenic mammalian milk producing FVII or FVIIa in its milk.

A method for producing a recombinant protein in the milk of a transgenic animal may include the following steps: A synthetic DNA molecule containing a gene that codes for a desired protein, which gene is controlled by a promoter of a protein that is naturally secreted in milk, is transferred into an embryo of a non-human mammal. The embryo is then introduced into a female mammal of the same species, which then gives birth to a transgenic animal. Once the subject is sufficiently developed, lactation of the mammal is induced and the milk is collected. The milk then contains the desired recombinant protein. One example of a process for the preparation of a protein in the milk of a female mammal other than a human being is provided in document No. EP 0 527 063, whose teaching may be applied to the production of the desired protein according to the invention.

A plasmid containing the WAP promoter is constructed through the introduction of a sequence containing the promoter for the WAP gene, and this plasmid is created in such a way that it can receive a foreign gene that is rendered dependent upon the WAP promoter. The gene that codes for a desired protein is incorporated and rendered dependent upon the WAP promoter. The plasmid containing the promoter and the gene that codes for the desired protein are used to obtain transgenic animals, such as rabbits, via microinjection into the male pronucleus of rabbit embryos. The embryos are then transferred to the oviduct of hormonally prepared females. The presence of the transgenes is detected via Southern blotting, using DNA extracted from the young transgenic rabbit produced. The concentrations in the animals' milk are evaluated using specific radioimmunological assays. Other documents describe methods for preparing a protein in the milk of a female mammal other than a human (U.S. Pat. No. 7,045,676 and EP 1 739 170—production of von Willebrand Factor in transgenic mammal).

In another particular embodiment, the sample is a sample containing purified FVIIa, in liquid or lyophilized form. For example, the purity of such a FVIIa is at least 80% and especially of at least 95%, or even 99%. In particular the sample is a therapeutic sample containing purified FVIIa in liquid or lyophilized form. With the method of the invention it is possible to measure the concentration of plasma FVIIa (pFVIIa), recombinant FVIIa (rFVIIa) or transgenic FVIIa (TgFVIIa).

Therefore, according to one particular embodiment, the invention concerns a method for the in vitro or ex-vivo measurement of the FVIIa concentration in a sample of transgenic mammalian milk, comprising the steps consisting of:

a) mixing said sample with a human plasma deficient in FVII and in at least one other factor chosen from among FVIII, FIX and FXI;

b) adding components to initiate the thrombin generation reaction, comprising a source of calcium ions, a phospholipid agent and tissue factor;

c) conducting a thrombin generation test (TGT) on the reaction medium obtained, to plot a thrombogram providing parameters;

d) comparing at least one of the parameters of the thrombogram with a homologous parameter of standard thrombograms, each standard thrombogram being obtained with a fixed, calibrated concentration of FVIIa in said reaction medium, lying in the range of 1 pM to 5 nM, and

e) inferring from step d) the measurement of the FVIIa concentration of the sample, lying within said range.

According to another particular embodiment, the invention concerns a method for the in vitro or ex-vivo measurement of the FVIIa concentration of a sample containing purified FVIIa, comprising the steps consisting of:

a) mixing said sample with a human plasma deficient in FVII and in at least one other factor chosen from among FVIII, FIX and FXI;

b) adding components to initiate the thrombin generation reaction, comprising a source of calcium ions, a phospholipid agent and tissue factor;

c) conducting a thrombin generation test (TGT) on the reaction medium obtained, to plot a thrombogram giving parameters;

d) comparing at least one of the parameters of the thrombogram with a homologous parameter of standard thrombograms, each standard thrombogram being obtained with a fixed, calibrated concentration of FVIIa in said reaction medium, lying in a range of 1 pM to 5 nM, and e) inferring from step d) the measurement of the FVIIa concentration of the sample, lying within said range.

The term <<deficient in>> means that the concentration of FVII, FVIII, FIX and/or FXI is below the threshold detection of said factor as measured by conventional assay methods well known to those skilled in the art. For example, kits or reagents available on the market may be used to perform conventional immunoassays. With the method of the invention it is possible to measure the concentration of plasma FVIIa, recombinant FVIIa and/or transgenic FVIIa.

According to one preferred embodiment, the plasma used in the method of the invention is deficient in FVII and in at least one other factor chosen from among FVIII, FIX and FXI. The human plasma deficient in FVII and in at least one other factor chosen from among FVIII, FIX and FXI can be obtained in different manners. In the method of the invention, it is possible to use a plasma of a haemophilia A individual for example (deficient in FVIII) which is then depleted of FVII, a plasma of a haemophilia B individual (deficient in FIX) which is then depleted of FVII, or a plasma of a patient having complete factor XI deficiency, which is then depleted of FVII.

According to one particular embodiment, the method of the invention uses a human plasma deficient in FVII and in at least one other factor chosen from among FVIII, FIX or FXI, which is an immunodepleted human plasma. Immunodepletion can be obtained in different manners e.g.
The plasma deficient in FVII and FVIII is obtained for example by depletion using specific antibodies of FVII, FVIII or a protein bound to one of these factors such as, but not being limited thereto, anti-FVII, anti-FVIII, anti-Willebrand factor, the Willebrand factor being a plasma protein which transports FVIII in the blood,
The plasma deficient in FV and FIX is obtained for example by depletion using specific antibodies of FVII, of FIX,
The plasma deficient in FVII and FXI is obtained for example by depletion using specific antibodies of FVII, of FXI or a protein bound to one of these factors such as anti-FVII and anti-FXI for example.

According to one particular embodiment of the invention, the human plasma deficient in FVII and in at least one other factor chosen from among FVIII, FIX and FXI is a chemically depleted human plasma. The human plasma can be depleted of FVIII, which is a Ca²⁺ dependent factor, using EDTA. Once the plasma has been depleted of FVIII, the EDTA is removed using methods well known to those skilled in the art, in particular by dialysis. The depletion methods can be combined together to obtain the plasma depleted of the above factors.

Preferably, at step b), the final concentration of calcium ions in the reaction medium lies between 14 and 18 mM, in particular it is 16.7 mM. The source of calcium ions represents any biologically compatible source, such as CaCl₂. The final concentration of the phospholipid agent in the reaction medium lies between 1 and 20 μM, in particular between 3 and 5 μM, and that of the tissue factor (TF) lies between 0.1 and 10 pM, in particular between 0.1 and 6 pM.

In one particular embodiment of the invention, the TF concentration to implement the method for the in vitro or ex vivo measurement of FVIIa concentration in a sample of purified FVIIa, lies between 0.1 and 10 pM, in particular between 1 and 5 pM. A further subject-matter of the present invention concerns the use of a human plasma deficient in factor VII and in at least one other factor chosen from among factor VIII, factor IX and factor XI for the in vitro or ex vivo measurement of factor VIIa concentration in a sample. As shown previously, it is the fact that a plasma is deficient not only in factor FVII but also in at least one of the above factors which makes it possible to determine a correlation between the quantity of FVIIa present in a sample and certain TGT parameters, which was hitherto unknown. According to one preferred embodiment of the invention, the sample whose FVIIa concentration is to be measured is a plasma sample, a therapeutic sample or a therapeutic sample containing recombinant or transgenic FVIIa, such as defined above.

A further subject-matter of the invention is a kit which may be used to implement the method of the invention, comprising:
a lyophilized plasma deficient in FVII and in at least one other factor chosen from among FVIII, FIX and FXI;
a lyophilisate containing a phospholipid agent and tissue factor; a source of calcium ions; and
a sample of lyophilised FVIIa to determine at least one of the parameters of standard thrombograms.
   Said kit is therefore advantageously used for implementing the method to measure the FVIIa concentration in a sample, according to the invention. For this purpose, it is nevertheless necessary to be equipped with a device to conduct the TGT test.

The device to conduct the thrombin generation test notably comprises a thrombin calibrator, available on the market, and a developing agent for the generated thrombin e.g. a suitable fluorogenic reagent, measurement apparatus e.g. a fluorimeter also comprising software adapted to collect data which can be used to plot a thrombogram, and microplates. Advantageously, the kit comprises recipients intended to contain the different lyophilisates. The content of said recipients is then dissolved in an aqueous solvent, such as water for injection (WFI), so as to obtain effective concentrations of the components of interest for conducting the TGT test. Said recipients may be the wells of microplates. Preferably, the final concentrations of the components of interest in the reaction medium are those indicated above. Preferably, the kit also comprises sampling devices, such as pipettes or micropipettes.

As mentioned above, to implement the method according to a preferred embodiment, a lyophilized plasma deficient in FVII and in at least one other factor chosen from among FVIII, FIX and FXI, and the lyophilisate containing a phospholipid agent, tissue factor and a source of calcium ions are dissolved in water for injection WFI to which different quantities of lyophilized FVIIa are added so that the final concentration of FVIIa in the reaction medium that is obtained lies between 1 pM and 5 nM.

In one particular embodiment, the invention concerns a kit which can be used to implement the method of the invention for the in vitro or ex vivo measurement of the FVIIa concentration in a sample of transgenic mammalian milk, which comprises:
a lyophilized plasma deficient in FVII and in at least one other factor chosen from among FVIII, FIX and FXI;
a lyophilisate containing a phospholipid agent and tissue factor;—a source of calcium ions; and
a lyophilized FVIIa sample to determine at least one of the parameters of standard thrombograms.

In one particular embodiment, the invention concerns a kit which can be used to carry out the method of the invention for the in vitro or ex vivo measurement of the FVIIa concentration in a sample containing purified FVIIa, which comprises:
a lyophilized plasma deficient in FVII and in at least one other factor chosen from among FVIII, FIX and FXI;
a lyophilisate containing a phospholipid agent and tissue factor;
a source of calcium ions; and
a sample of lyophilized FVIIa to determine at least one of the parameters of standard thrombograms.

A further subject of the invention concerns the use of a kit such as defined previously for the in vivo, in vitro or ex vivo measurement of the FVIIa concentration in a sample. Said kit is easy to use and can be stored at a temperature of 4° C. for at least 1 year. The only equipment required is a developing agent for the generated thrombin e.g. a suitable fluorogenic reagent as defined above, measurement apparatus e.g. a fluorimeter comprising software allowing a thrombogram to be plotted, and microplates to conduct the TGT test in order to determine accurately the unknown FVIIa concentration of a sample. This assumes the plotting of standard thrombograms and a thrombogram of the sample whose FVIIa concentration it is desired to determine. According to one particular embodiment, the kit is used to measure the FVIIa concentration of a therapeutic sample or not, containing plasmatic, recombinant or transgenic FVIIa, such as defined above.

### DETAILED DESCRIPTION

The examples given below illustrate the invention without the limiting the scope thereof.

### Examples

### Example 1

### Obtaining A FVII-Deficient Plasma

A polyclonal antibody produced in rabbit directed against purified human plasma FVII, was coupled with rCNB-activated Sepharose (Pharmacia), then 2 mL of the gel obtained were placed in a column. The column was equilibrated with 25 mL equilibrating buffer (0.15 M NaCl, 10 mM citrate, pH 7.4). Next, 6 mL of human plasma were passed through the column. Under these conditions, the FVII remains fixed to the column and the eluate is collected. The column was regenerated eluting the fixed FVII with 20 mL of regeneration buffer (50 mM NaCl; 0.1 M glycine, pH 2.4) then the column was re-equilibrated with 20 mL of equilibrating buffer (10 mM citrate; 0.15 M NaCl, pH 7.4). These steps were repeated 7 to 8 times to obtain 6 mL of FVII-deficient plasma.

### Example 2

### Preparation of the Reaction Medium to Plot a Standard Thrombogram

An adequate volume of an rFVIIa sample (NovoSeven®-Novonordisk) is taken, which is added to 80 μl of FVII-deficient plasma obtained in Example 1, so that each reaction mixture contains a final calibrated concentration of between 0.02 nM and 10 nM. The reaction medium is obtained by mixing 20 μl of initiating factors of the thrombin generation reaction (Ca²⁺, phospholipids and TF) having final concentrations of 5 pM TF, 4 μM phospholipids (Biodis TS 30.00 reagent-Biodis) and 16.7 mM Ca²⁺, and 20 μl of thrombin-specific fluorogenic agent (Fluca reagent kit Biodis TS 50.00) with the above plasma containing rFVIIa.

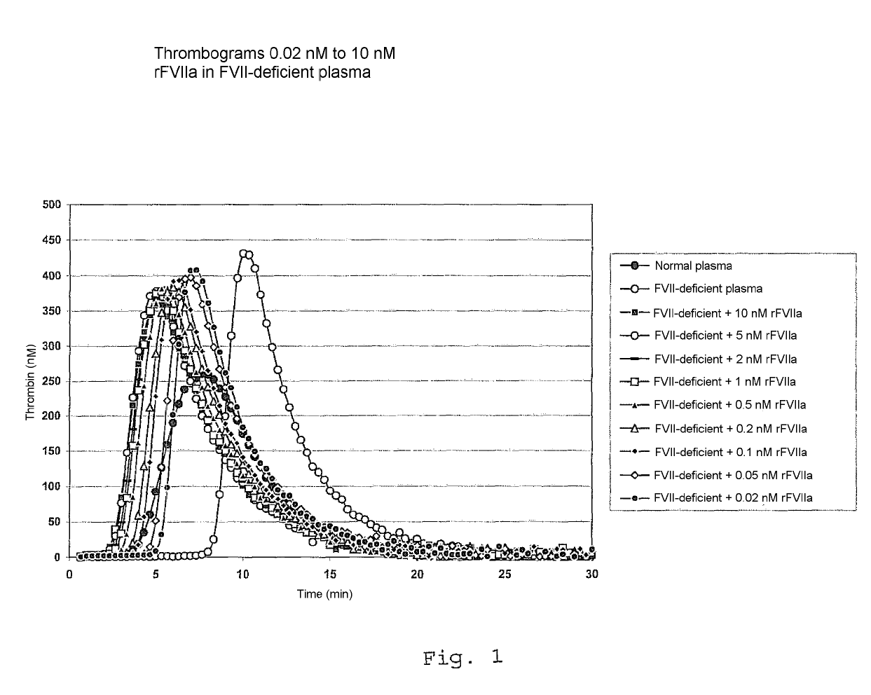

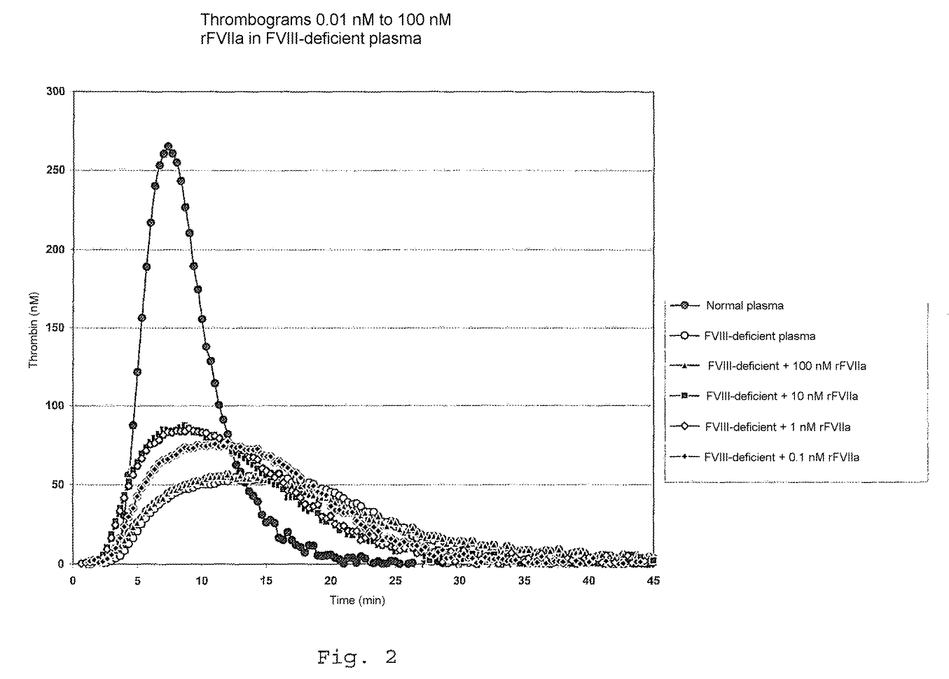

The TGT curves were plotted for final concentrations of rFVIIa lying between 0.02 nM and 10 nM, so as to obtain the thrombograms shown in FIG. 1. They were plotted using a fluorometer device measuring thrombin formation time (Fluoroskan-Thermo Electron) equipped with software to plot thrombograms (Thrombinoscope BV), at an excitation wavelength of 390 nm and detection at an emission wavelength of 460 nm. This figure shows that the different thrombograms determined for the plasma deficient in FVII alone, do not allow a correlation to be obtained between the rFVIIa concentration and the thrombin formation time measured at the peak. Indeed no notable variation is observed between this time-to-peak and the quantity of rFVIIa, nor any significant variation in peak heights in relation to this quantity.

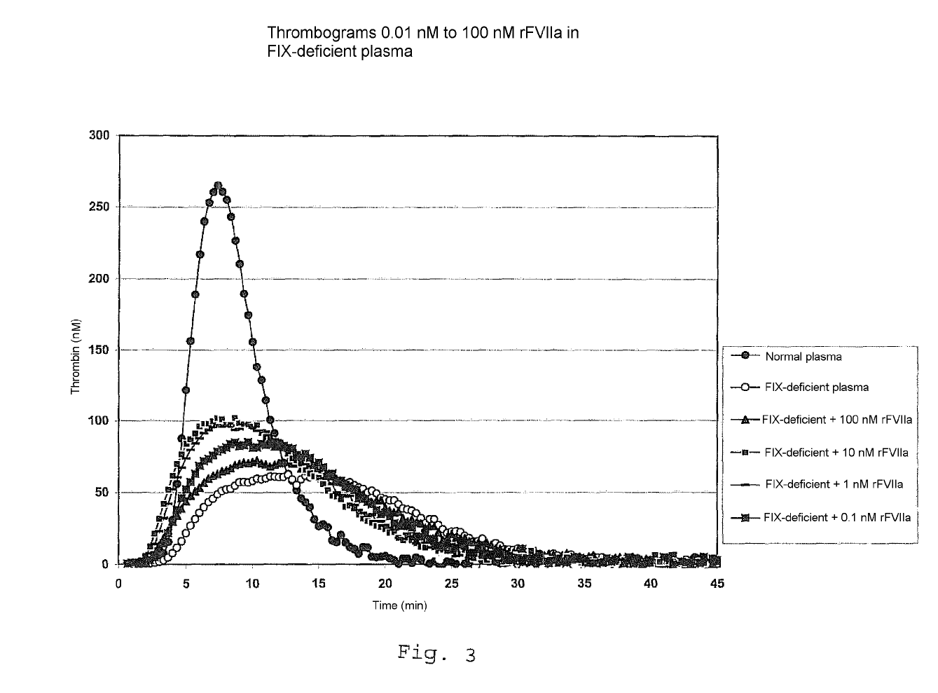

FIGS. 2 and 3 show the respective thrombograms obtained by adding 0.1 nM to 100 nM rFVIIa to the plasma deficient in FVIII alone, acquired from Diagnostica Stago or from haemophilia A individuals, and deficient in FIX alone acquired from Diagnostica Stago, respectively. These figures show that the different thrombograms plotted for the plasmas deficient in respectively either FVIII alone or FIX alone, do not allow a correlation to be obtained between FVIIa concentration and peak thrombin formation time. No notable variation is observed in this time-to-peak, i.e. it is practically constant in relation to the quantity of added FVIIa.

### Example 3

### Obtaining a plasma Deficient in FVII and in FVIII, FIX or FXI

A polyclonal antibody produced in rabbit directed against purified human plasma FVII was coupled with rCNB-activated Sepharose (Pharmacia), then 2 mL of the gel obtained was placed in a column. The column was equilibrated with 25 mL equilibrating buffer (0.15 M NaCl, 10 mM citrate, pH 7.4). Next, 6 mL of commercially available plasma already depleted of FVIII, FIX or FXI, each acquired from Diagnostica Stago, were passed through the column. Under these conditions, the FVII remains fixed to the column and the eluate is collected. The column was regenerated eluting the fixed FVII with 20 mL regeneration buffer (50 mM NaCl; 0.1 M glycine, pH 2.4) then the column was re-equilibrated with 20 mL equilibrating buffer (10 mM citrate; 0.15 M NaCl; pH 7.4). These steps were repeated 7 to 8 times to obtain 6 mL of doubly depleted plasma, deficient in FVIII, FIX or FXI and in FVII.

### Example 4

### Preparation of the Reaction Medium to Plot a Standard Thrombogram

An adequate volume of an rFVIIa sample is taken, which is added to 80 μl of FVIII-deficient plasma acquired from Diagnostica Stago or from haemophilia A individuals, which was then depleted of FVII following the procedure in Examples 1 and 3, so that each reaction mixture contains a final calibrated concentration of rFVIIa of between 1 pM and 1 nM (is it not rather 5 nM). The reaction medium is obtained by mixing 20 μl of initiating factors of the thrombin generation reaction (Ca²⁺, phospholipids and TF) having final concentrations of 5 pM TF, 4 μM phospholipids (Biodis TS 30.00 reagent-Biodis) and 16.7 mM Ca²⁺, and 20 μl of thrombin-specific fluorogenic agent (Fluca reagent kit Biodis TS 50.00) with the above FVIIa-containing plasma.

The TGT curves were plotted for final concentrations of rFVIIa lying between 1 pM and 5 nM, so as to obtain thrombograms whose various parameters (lag time, peak height, time to peak and velocity) are progressively corrected, dose-dependent fashion, in relation to the FVIIa content. They are plotted using the same fluorometer measurement device and under the same conditions as for Example 2.

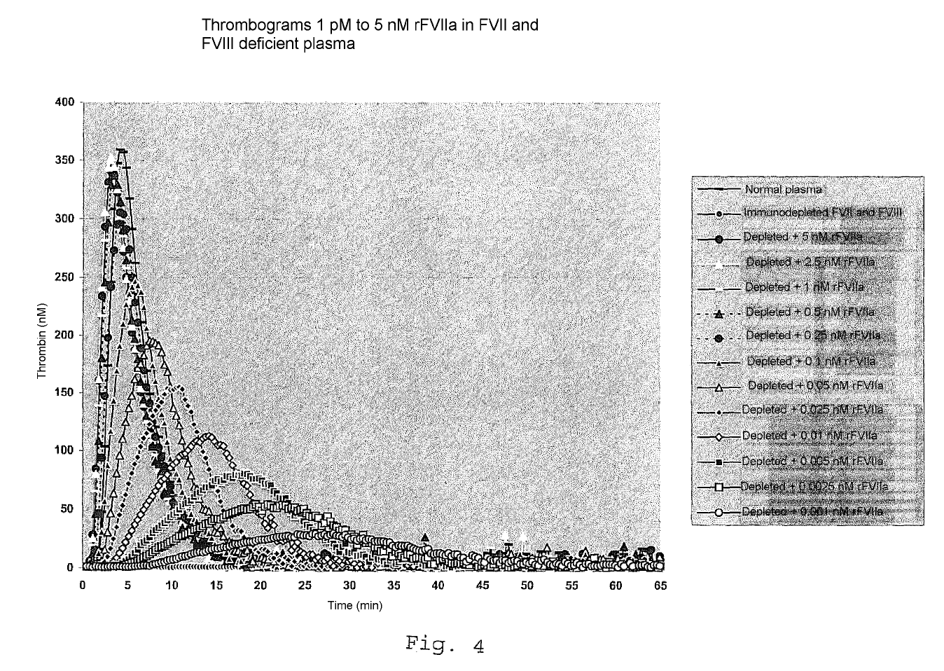

FIG. 4 shows the thrombograms obtained in the presence of 1 pM to 5 nM rFVIIa in plasma deficient in FVII and FVIII. A reduction in peak thrombin formation time is observed in relation to the quantity of rFVIIa present in the sample. This time reaches a limit which can be estimated at 3 minutes at a rFVIIa concentration of 1 nM. It is noted that a plasma deficient in FVII and in FVIII does not generate any thrombin formation.

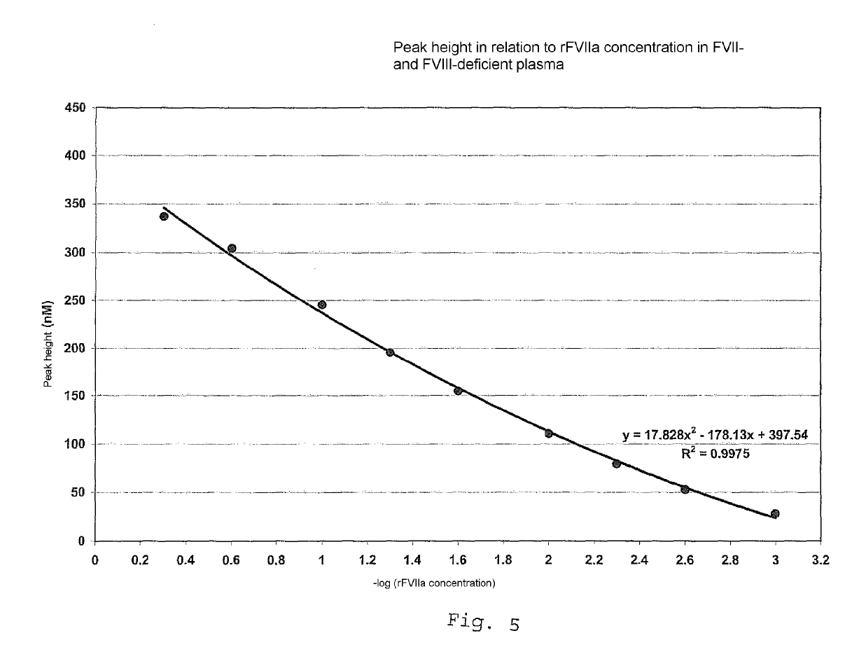

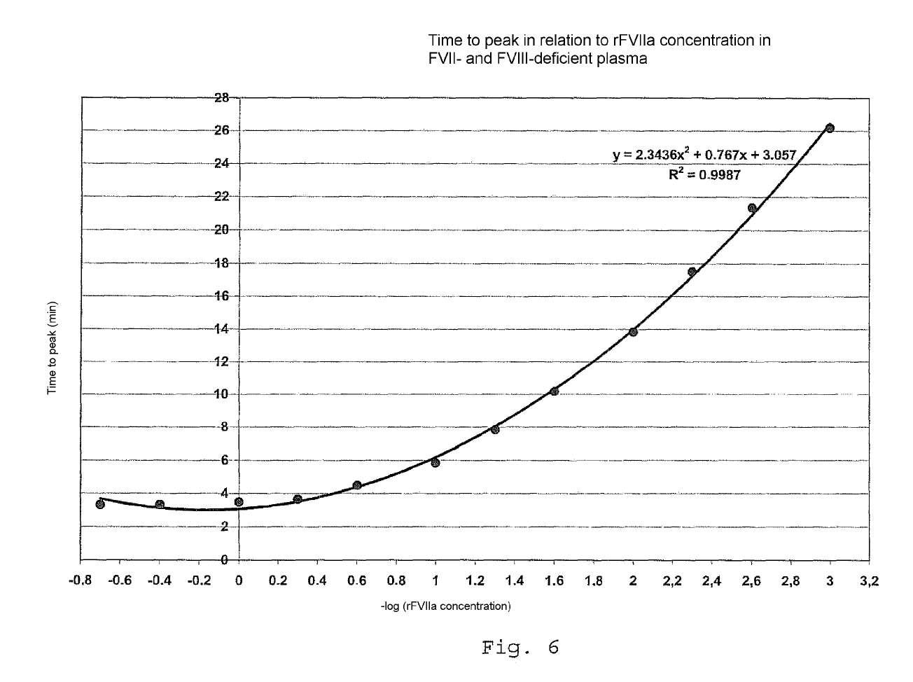

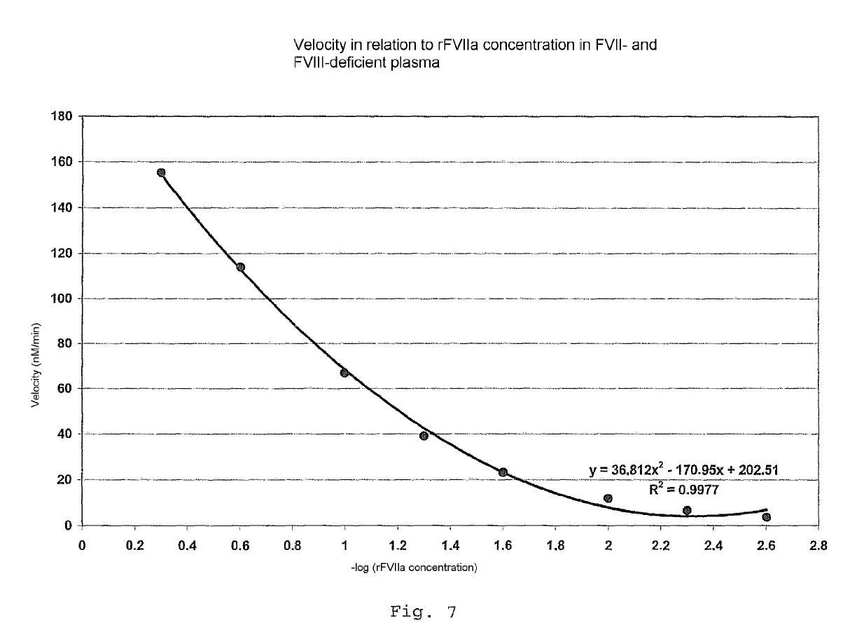

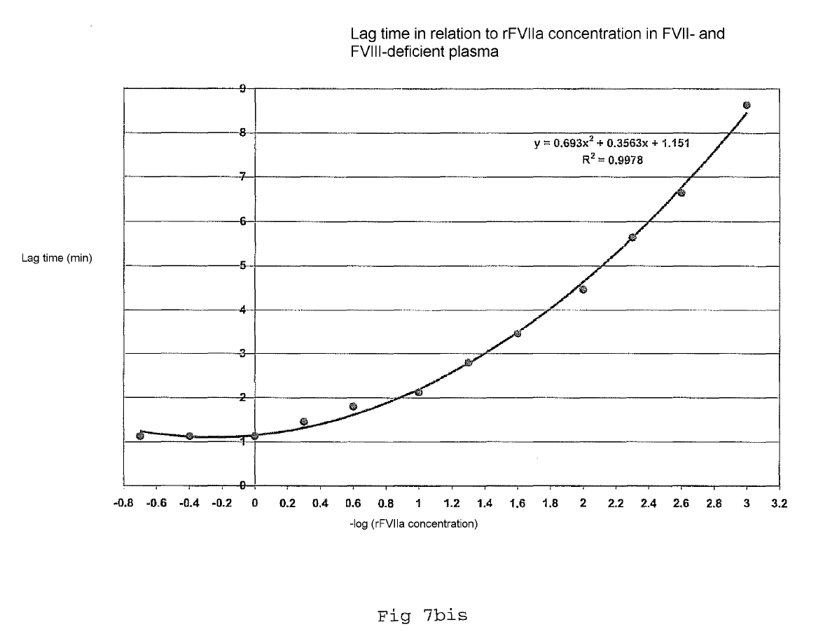

FIGS. 5, 6, 7 and 7bis respectively show the variations in peak heights, time to peak, velocity and lag time, in relation to the quantity of rFVIIa present in the sample and hence in the reaction medium. The results obtained show that it is possible to establish a correlation between FVIIa concentration and the different parameters deduced from thrombograms at between 1 pM and 5 nM. The same experiment was conducted with a TF concentration of 1 pM (results not shown) (give a short conclusion for this case).

### Example 5

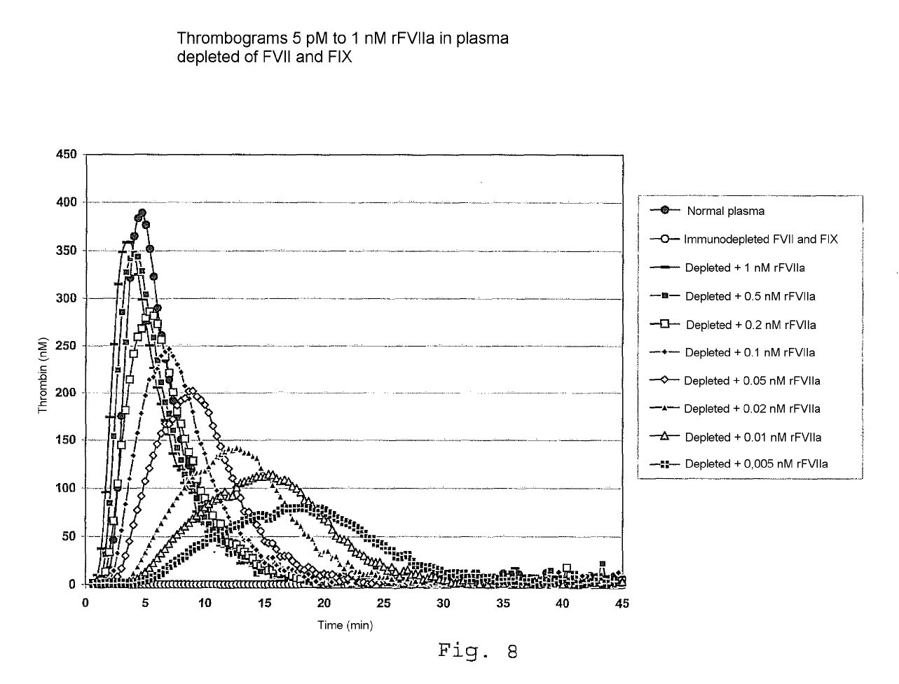

The experiment in Example 4 was repeated, but considering a reactive plasma deficient in FIX, acquired from Diagnostica Stago, which was then depleted of FVII following the procedure in Examples 1 and 3. FIG. 8 shows the thrombograms obtained in the presence of 5 pM to 1 nM rFVIIa in plasma deficient in FVII and in FIX. A reduction in the peak thrombin formation time is observed, in relation to the quantity of FVIIa present in the sample. This time also reaches a limit which can be estimated at 3 minutes at a rFVIIa concentration of 1 nM. It is noted that a plasma deficient in FVII or FIX without the addition of FVIIa does not generate any thrombin formation.

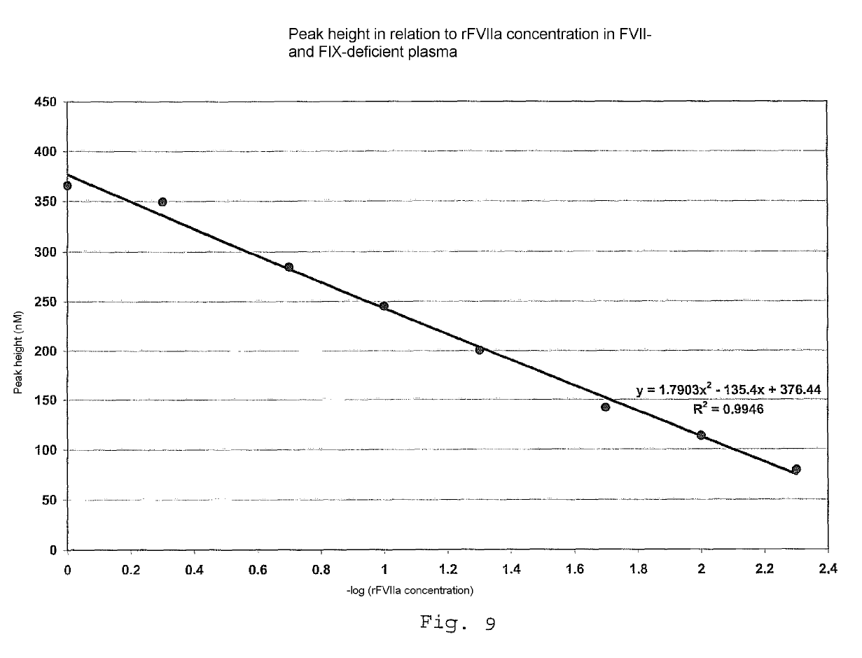

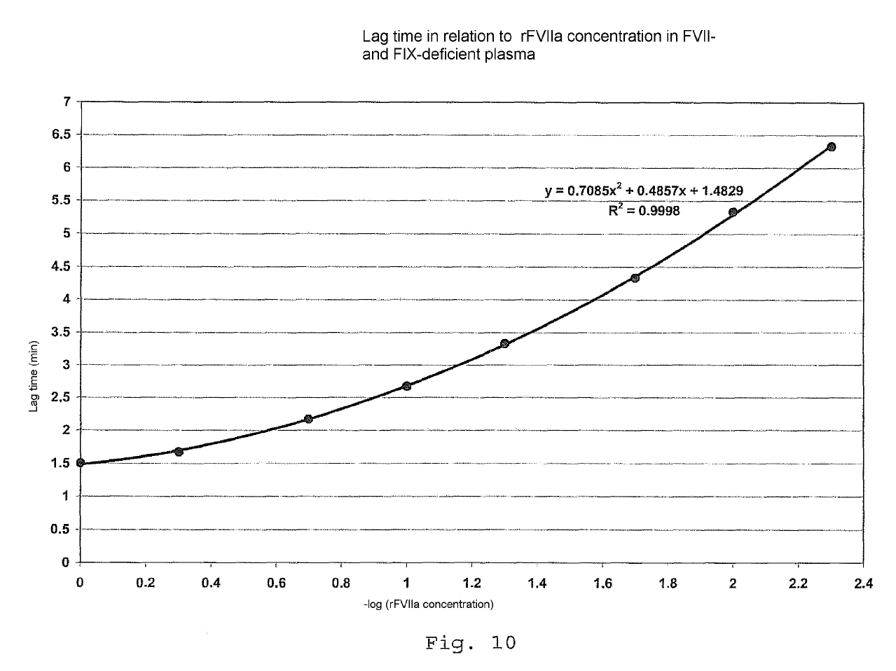

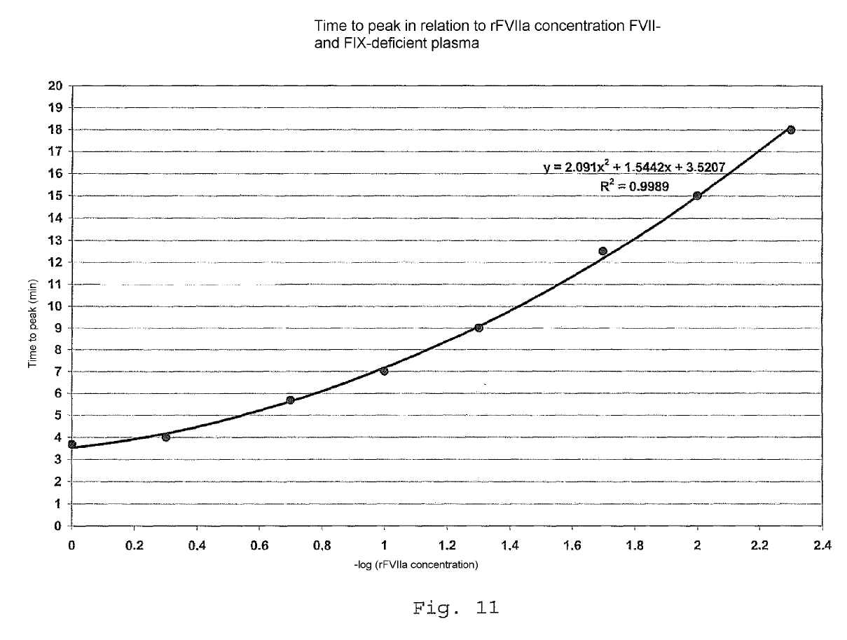

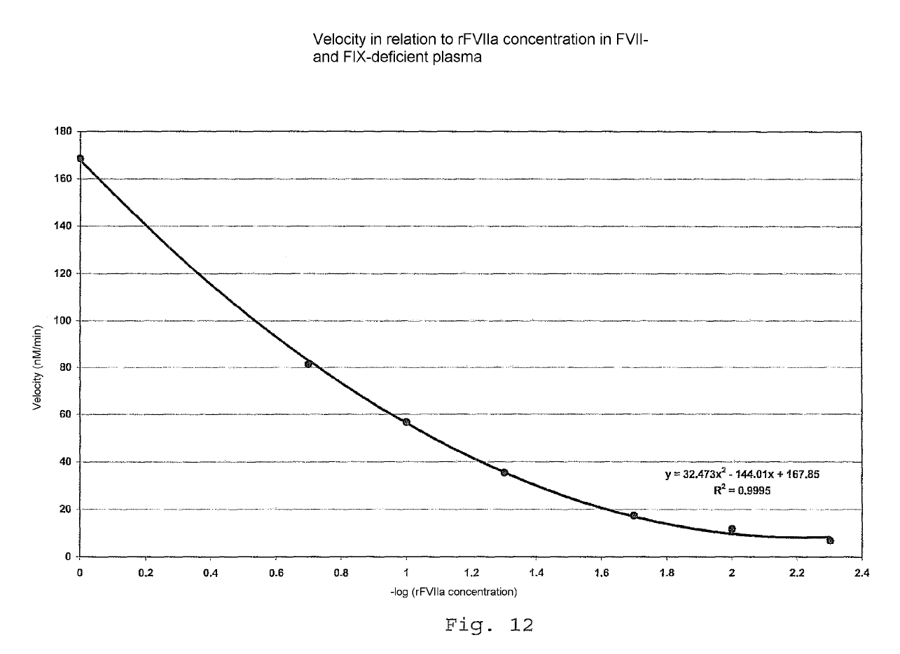

FIGS. 9, 10, 11 et 12 respectively illustrate the variations in peak heights, lag times, times to peak and velocity, in relation to the quantity of rFVIIa present in the sample and hence in the reaction medium. The results obtained show that it is possible to establish a correlation between FVIIa concentration and the different parameters deduced from said thrombograms.

### Example 6

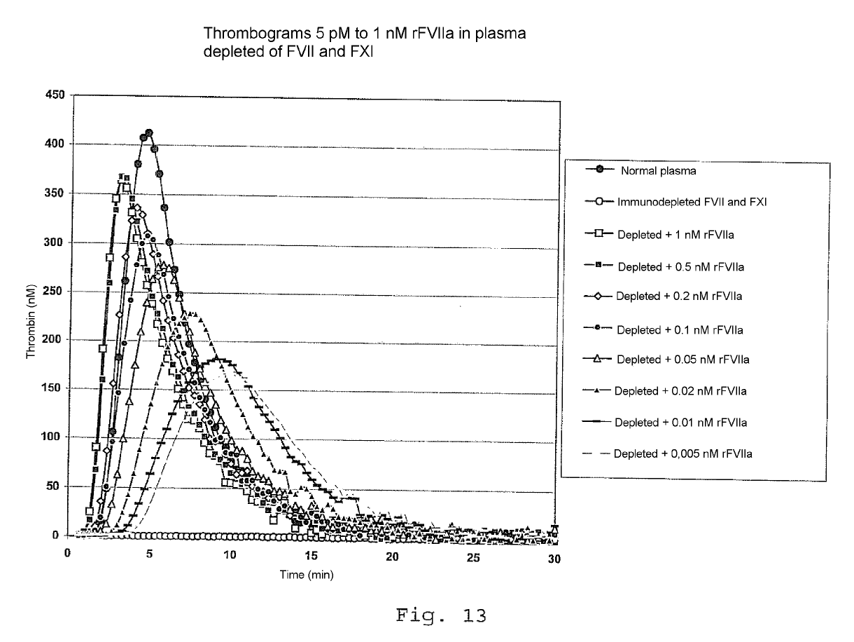

The experiment in Example 4 is repeated, but considering a reactive plasma deficient in FXI, derived from Diagnostica Stago, which was then depleted of FVII following the procedure in Examples 1 and 3. FIG. 13 shows the thrombograms obtained in the presence of 5 pM to 1 nM rFVIIa in plasma deficient in FVII and FXI. A reduction in the peak thrombin formation time is observed in relation to the quantity of FVIIa present in the sample. It is noted that a plasma deficient in FVII and in FXI does not generate any thrombin formation.

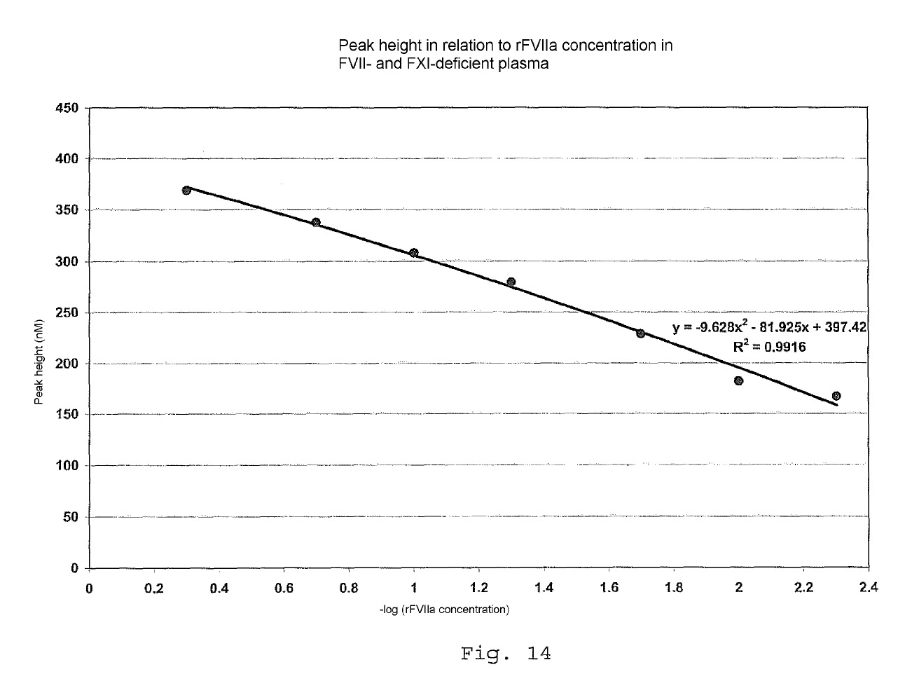

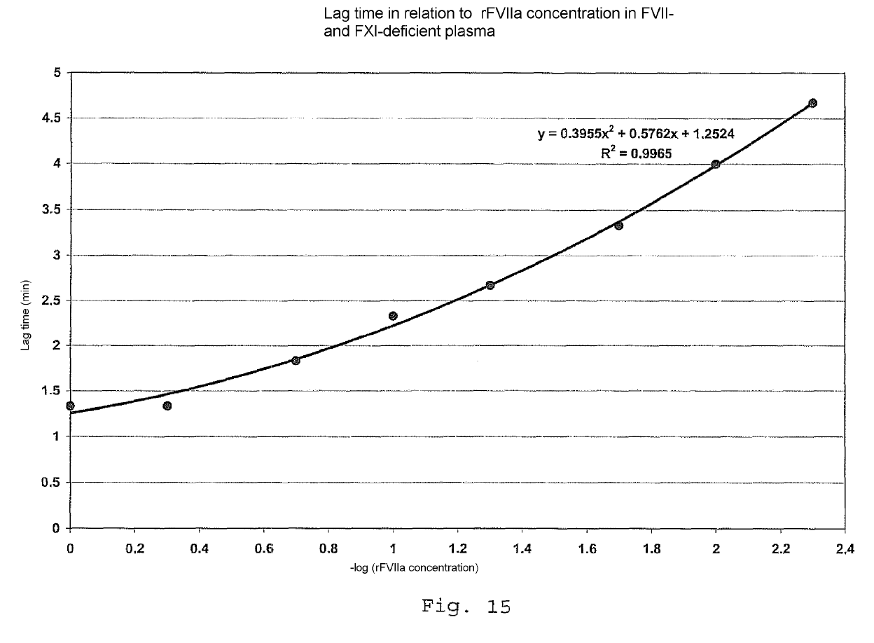

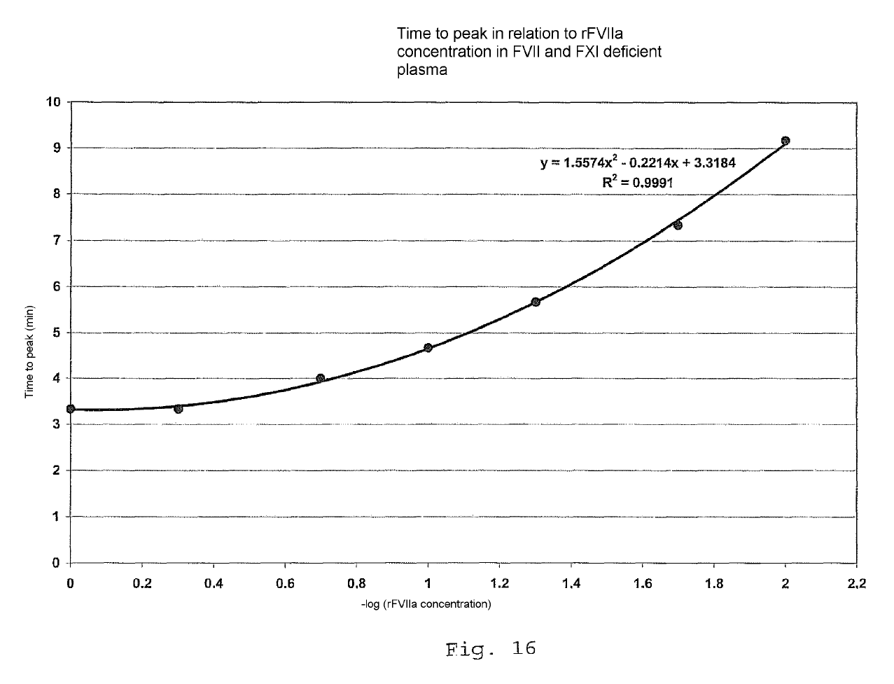

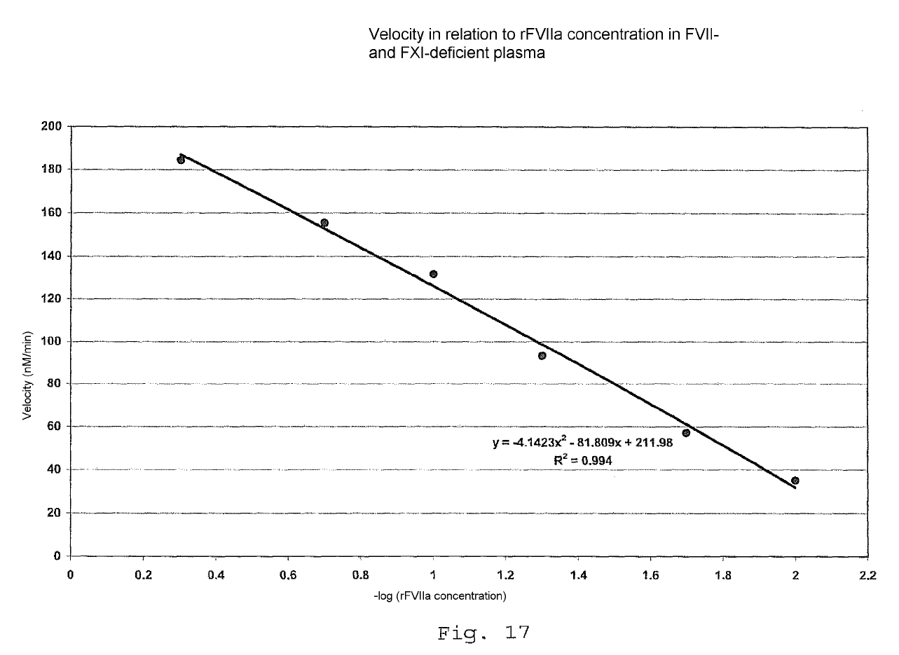

FIGS. 14, 15, 16 and 17 respectively show the variations in peak heights, lag time, time to peak and velocity, in relation to the quantity of rFVIIa present in the sample and hence in the reaction medium. The results obtained show that it is possible to establish a correlation between FVIIa concentration and the different parameters deduced from the thrombograms.

### Example 7

### Example Measurement of FVIIa Concentration in a Sample of Unknown Concentration

A volume of 10 μl of a sample having an unknown rFVIIa concentration, was mixed with plasma depleted of FVII and FVIII, such as described previously. Components initiating the thrombin generation reaction were added (Ca²⁺, phospholipids and TF) at final concentrations of 5 pM FT, 4 μM phospholipids (Biodis reagent TS 30.00), 16.7 mM Ca⁺², as well as 20 μl of thrombin-specific fluorogenic agent (Fluca reagent kit Biodis TS 50.00). Different dilutions of the rFVIIa sample were made (see Table 1) and a constant volume of 10 μl of these diluted samples were added to 170 μl of plasma depleted of FVII and of FVIII to obtain a total volume of 200 μl of reaction medium. A TGT test was performed for the different dilutions of the sample (see Tableau 1) to obtain thrombograms and the corresponding different parameters: lag time, peak height, time to peak and velocity.

In parallel, different samples of 4 μl of known rFVIIa calibrated concentration were prepared which were mixed with 76 μl of plasma depleted of FVII and FVIII, so as to obtain final rFVIIa concentrations of between 1 pM and 5 nM for a volume of 80 μl. Initiator factors of thrombin generation reaction were added (Ca²⁺, phospholipids and TF) at final concentrations of 5 pM FT, 4 μM phospholipids (Biodis reagent TS 30.00), 16.7 mM Ca⁺² together with 20 μl of thrombin-specific fluorogenic agent (Fluca reagent Biodis kit TS 50.00). The parameters of standard thrombograms were measured to trace calibrated curves of the values of each of the parameters in relation to the −log of the final rFVIIa concentration (see FIGS. 5, 6, 7 and 7bis).

The values of the parameters obtained for the different dilutions of the sample of unknown rFVIIa concentration were plotted over the different standard calibrated curves. The dilutions 1/200 to 1/5000 correspond to respective concentrations on the calibrated curves of 0.25 nM to 0.01 nM. Giving consideration to the dilution factor, the rFVIIa concentration of the unknown sample is 50 nM.

TABLE 1
Value Of The Different Parameters For The Sample Of Unknown FVIIa Concentration

| | Dilution | Dilution | Dilution | Dilution | Dilution | Dilution | Dilution | Dilution | Dilution |
|---|---|---|---|---|---|---|---|---|---|
| Parameters | 1/10 | 1/20 | 1/50 | 1/100 | 1/200 | 1/500 | 1/1000 | 1/2000 | 1/5000 |
| Lag time | 1.13 | 1.13 | 1.13 | 1.46 | 1.8 | 2.13 | 2.8 | 3.47 | 4.47 |
| Peak | 343.27 | 352.23 | 345.72 | 336.76 | 304.22 | 245.44 | 195.49 | 154.73 | 110.47 |
| height | | | | | | | | | |
| Time to | 3.3 | 3.3 | 3.47 | 3.63 | 4.47 | 5.81 | 7.81 | 10.15 | 13.82 |
| peak | | | | | | | | | |
| Velocity | 158.19 | 162.31 | 147.74 | 155.18 | 113.94 | 66.69 | 39.01 | 23.16 | 11.81 |

1. Method for the in vitro or ex-vivo measurement of the concentration of factor VIIa (FVIIa) in a sample, comprising the step consisting of:
a) mixing said sample with a human plasma deficient in FVII and in at least one other factor chosen from among factor VIII (FVIII), factor IX (FIX) and factor XI(FXI);
b) adding initiator components of the thrombin generation reaction, comprising a source of calcium ions, a phospholipid agent and tissue factor;
c) performing a thrombin generation test (TGT) on the reaction medium thus obtained to obtain a thrombogram giving parameters;
d) comparing at least one of the parameters of the thrombogram with a homologous parameter of standard thrombograms, each standard thrombogram being obtained with a fixed, calibrated concentration of FVIIa in said reaction medium, lying in a range of 1 pM to 5 nM, and
e) deducing from step d) the measurement of the FVIIa concentration of the sample, lying within said range.

2. Method according to claim 1, wherein the parameters of standard thrombograms are determined by conducting a series of thrombin generation tests TGT with standard samples, consisting of the same reaction medium, each of said samples containing a fixed, final calibrated concentration of FVIIa in the range 1 pM to 5 nM.

3. Method according to claim 1 or 2, characterized in that at step d) at least one of the parameters of the thrombogram is chosen from among peak height, velocity, lag time and time to peak.

4. Method according to any of claims 1 to 3, characterized in that the sample containing FVIIa is a sample of transgenic mammalian milk or a sample containing purified FVIIa.

5. Method according to any of claims 1 to 4, characterized in that the FVIIa is a plasma FVIIa (pFVIIa), a recombinant FVIIa (rFVIIa) or a transgenic FVIIa (TgFVIIa).

6. Method according to any of claims 1 to 5, characterized in that the human plasma deficient in FVII and in at least one other factor chosen from among FVIII, FIX and FXI, is a immunodepleted human plasma.

7. Method according to any of claims 1 to 5, characterized in that the human plasma deficient in FVII and in at least one other factor chosen from among FVIII, FIX and FXI is a chemically depleted human plasma.

8. Method according to claim 7, characterized in that the human plasma deficient in factor VIII is chemically depleted with EDTA.

9. Kit which can be used to implement the method such as defined in any of claims 1 to 8, comprising:
a lyophilized plasma deficient in FVII and in at least one other factor chosen from among FVIII, FIX and FXI;
a lyophilisate containing a phospholipid agent and tissue factor;
a source of calcium ions; and
a sample of lyophilized FVIIa of known concentration to determine at least one of the parameters of standard thrombograms.

10. Use of a kit such as defined according to claim 9, for the in vitro or ex-vivo measurement of the FVIIa concentration of a sample.

11. Use according to claim 10, characterized in that the sample is a sample of transgenic mammalian milk or a sample containing purified FVIIa.

12. Use according to either of claims 10 or 11, characterized in that the FVIIa is a plasma FVIIa (pFVIIa), a recombinant FVIIa (rFVIIa) or a transgenic FVIIa (TgFVIIa).

13. Use of human plasma deficient in FVII and in at least one other factor chosen from among FVIII, FIX and FXI for the in vitro or ex-vivo measurement of the FVIIa concentration of a sample.

14. Use according to claim 13, characterized in that the sample is a sample of transgenic mammalian milk or a sample containing purified FVIIa.

15. Use according to either of claims 13 or 14, characterized in that the FVIIa is a plasma FVIIa (pFVIIa), a recombinant FVIIa (rFVIIa) or a transgenic FVIIa (TgFVIIa).

16. Plasma deficient in Factor VII (FVII) and in at least one other factor chosen from among factor VIII (FVIIa), factor IX (FIX) and factor XI (FXI).

17. Method for preparing a plasma deficient in FVII and in at least one other factor chosen from among FVIII, FIX and FXI, comprising the chemical or immunological depletion of a starting plasma in at least one of the said factors FVII, FVIII, FIX and/or FXI, said immunological depletion being performed using specific antibodies directed against said at least one factor to be depleted or against a protein which binds to this at least one factor to be depleted.

18. Method according to claim 17, wherein said starting plasma is selected from the group comprising a human plasma, a plasma obtained from a type A hemophiliac, a plasma obtained from a type B hemophiliac, or a plasma obtained from a patient suffering from a total deficiency in factor XI.

19. Plasma obtainable by the method according to the claim 17 or 18.
